(19) United States
(12) Patent Application Publication
(10) Pub. No.: US 2009/0216534 A1
(43) Pub. Date: Aug. 27, 2009

(54) VOICE-ACTIVATED EMERGENCY MEDICAL SERVICES COMMUNICATION AND DOCUMENTATION SYSTEM

(76) Inventor: Prakash Somasundaram, Monroeville, PA (US)

Correspondence Address: WOOD, HERRON & EVANS, LLP, 2700 CAREW TOWER, 441 VINE STREET, CINCINNATI, OH, 45202, US

(21) Appl. No.: 12/389,443

(22) Filed: Feb. 20, 2009

Related U.S. Application Data
(60) Provisional application No. 61/030,754, filed on Feb. 22, 2008.

(51) Int. Cl. G10L 15/04; G06Q 50/00; G06Q 10/00

(52) U.S. Cl. 704/251; 705/2; 705/3; 705/28

(57) ABSTRACT

A method of documenting information as well as a documentation and communication system for documenting information with a wearable computing device of the type that includes a processing unit and a touchscreen display is provided. The method includes displaying at least one screen on the touchscreen display. A field on the screen in which to enter data is selected and speech input from a user is received. The speech input is converted to machine readable input and the machine readable input is displayed in the field on the at least one screen.

### CROSS-REFERENCE TO RELATED APPLICATIONS

This application is related to and claims the benefit of U.S. Provisional Patent Application Ser. No. 61/030,754 to Prakash Somasundaram, entitled “VOICE-ACTIVATED EMERGENCY MEDICAL SERVICES COMMUNICATION AND DOCUMENTATION SYSTEM” (WHE Ref: VOCO-106P) and filed on Feb. 22, 2008, which application is incorporated by reference herein.

### FIELD OF THE INVENTION

The present invention relates to converting speech input to machine readable input, and more particularly to the documentation of information with a wearable voice-activated communication and documentation system.

### BACKGROUND OF THE INVENTION

Emergency Medical Service Technicians (“EMT's”) typically function as a part of an EMT team overseen and managed by an Emergency Medical Service (“EMS”) agency. Each EMT team is typically comprised of two or more persons that are in turn assigned to an ambulance and dispatched to a location to care for one or more patients in need of medical assistance. The EMS agency will generally maintain a station or headquarters for centralized oversight and direction of multiple EMS teams. Each EMT team is typically comprised of two EMT's, or an EMT and a paramedic. Each EMT team typically documents the care of the patients and any other observations that are made at the scene, during transport to the hospital, during treatment of the patient, or for administrative purposes. This documentation is typically used to determine billing for the patient and/or hospital and ensure patient safety by providing a list of treatment and procedures performed on the patient.

The documentation aspect of each EMT team is typically performed to maintain appropriate records that can be submitted to the hospital, the EMS agency, a state repository, and/or any other entity that may need documentation of the work of the EMT team. Currently, documentation typically involves using many different documentation modes, including scratch notes, writing notes on the backs of hands and gloves, paper trip sheets, and clip boards that the EMT team uses to manually fill out a trip sheet. The trip sheet typically includes dispatch information, scene information, patient information, medications administered, procedures performed, and times associated with dispatch, patient, scene, medication, or procedure information. One copy of the trip sheet is generally provided to the hospital when the EMT team arrives with the patient, while another copy is taken back to the EMS agency.

The data from the trip sheet is typically manually entered by a nurse or other person at the hospital for subsequent distribution to the physicians or attendants that care for the patient. The data from each trip sheet is also typically manually entered by the EMT team into a computer at the EMS station for submission to the state and the hospital or to the patient for billing. Such documentation issues are compounded when there are multiple dispatches made without the EMT team being able to return to the EMS agency and fill out their various trip sheets. As such, the current documentation process occupies a large amount of time of each EMT team and hospital employees. The current process also generates redundant work through redundant data entry and form completion by multiple parties. Such procedures ultimately reduce the amount of time EMT teams are available for dispatch and calls. Recent documentation process improvements involve of the use of laptops or PDA's that can be carried in the ambulance. The EMT teams may be provided with electronic trip sheets to complete documentation. Despite these improvements, EMT teams must still use their hands to administer patient care. Therefore, such reporting tasks are laborious and time-consuming, and involve the use of hands where they are wearing gloves, dealing with immobile patients, administering fluids, and coping with infection safety. Therefore, trip sheets (electronic or paper) still remain incomplete until the end of each dispatch, especially when there are multiple dispatches by an EMT team without returning to the EMS agency to fill out trip sheets. In such an environment, where it is not ideal for using hand-held devices, laptops, or paper trip sheets, EMT's would tend to write on gloves, use scratch sheets, or try to remember most of the information to document later. After multiple trips, such information is often documented from memory, and can lead to significant inaccuracies and incompleteness.

Documentation is typically performed by the EMT teams before a dispatch as well. For example, the EMT teams are typically required to maintain a pre-shift checklist of the equipment in their ambulance and maintain documentation certifying their readiness. The EMT teams also generally document and account for all medication in the ambulance inventory. In this way, an excessive amount of time is also typically spent on preparative tasks to achieve a high readiness factor for dispatches.

In addition to the documentation issues, the EMT teams must typically communicate with various entities (i.e., hospitals, the EMS stations, law enforcement entities, state entities) through devices that they carry and use during a dispatch. These devices may be two-way radios, pagers, and cell phones. However, there is typically no standardized communication system in an area that is adopted by all the entities that the EMT teams may have to contact.

Still further, each EMT team is typically provided with various paper documents that outline treatment protocol, procedure references, contraindications lists, and other paper-based information that may be needed to treat patients. The various paper documents and references not only take up space in the ambulance, but also may be difficult to refer to when treating the patient in a moving vehicle, as may be appreciated.

Consequently, there is a need for a system to document a dispatch, communicate with various entities, refer to documentation, and otherwise manage information and services to increase the efficiency, accuracy, readiness and availability of emergency medical services.

### SUMMARY OF THE INVENTION

Embodiments of the invention provide a method of documenting information as well as a documentation and communication system for documenting information. In some embodiments, the method includes a wearable computing device of the type that includes a processing unit and a touchscreen display. The method includes displaying at least one screen on the touchscreen display. A field on the screen in which to enter data is selected and speech input from a user is received. The speech input is converted to machine readable input and the machine readable input is displayed in the field on the at least one screen.

These and other advantages will be apparent in light of the following figures and detailed description.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the principles of the invention.

FIG. 1 is a diagrammatic illustration of an overview of a hardware environment for a documentation and communication system consistent with embodiments of the invention;

FIG. 2 is a diagrammatic illustration of a body unit and headset of the documentation and communication system of FIG. 1;

FIG. 3 is a diagrammatic illustration of a plurality of software components of the body unit of FIG. 2;

FIG. 4 is a diagrammatic illustrating of a hardware and software environment of a computing device to receive trip data consistent with embodiments of the invention;

FIG. 5 is a flowchart illustrating a sequence of steps during which a user may be dispatched to a patient to render transport for and/or emergency medical services to that patient consistent with embodiments of the invention;

FIG. 6 is a flowchart illustrating a sequence of steps to enter trip data that may be converted with an extended library with the body unit of FIG. 1;

FIG. 7 is a flowchart illustrating a sequence of steps to enter trip data that may be converted with a limited library with the body unit of FIG. 1;

FIG. 8 is a diagrammatic illustration of a call response screen that may be displayed by the body unit of FIG. 1;

FIG. 9 is a diagrammatic illustration of an incident location screen that may be displayed by the body unit of FIG. 1;

FIG. 10 is a diagrammatic illustration of an assessment screen that may be displayed by the body unit of FIG. 1;

FIG. 11 is a diagrammatic illustration of a patient information screen that may be displayed by the body unit of FIG. 1;

FIG. 12 is a diagrammatic illustration of a medical history screen that may be displayed by the body unit of FIG. 1;

FIG. 13 is a diagrammatic illustration of a patient disposition screen that may be displayed by the body unit of FIG. 1;

FIG. 14 is a diagrammatic illustration of a narrative screen that may be displayed by the body unit of FIG. 1;

FIG. 15 is a diagrammatic illustration of a notes screen that may be displayed by the body unit of FIG. 1;

FIG. 16 is a diagrammatic illustration of a vitals screen that may be displayed by the body unit of FIG. 1;

FIG. 17 is a diagrammatic illustration of a times screen that may be displayed by the body unit of FIG. 1;

FIG. 18 is a diagrammatic illustration of a procedures screen that may be displayed by the body unit of FIG. 1;

FIG. 19 is a diagrammatic illustration of a medications screen that may be displayed by the body unit of FIG. 1;

FIG. 20 is a flowchart illustrating a sequence of operations that may be performed by the body unit of FIG. 1 to display images and/or a multimedia presentation, and/or play audio prompts, of a protocol and/or procedure;

FIG. 21 is a flowchart illustrating a sequence of operations that may be performed by the body unit of FIG. 1 to determine whether, upon start-up or upon a request from a user, there is a portion of the inventory that is too low or unavailable;

FIG. 22 is a flowchart illustrating a sequence of operations that may be performed by the body unit of FIG. 1 to determine whether, upon use of a piece of the inventory or and indication that a piece of inventory is unavailable, that portion of the inventory is too low or unavailable;

FIG. 23 is a flowchart illustrating a sequence of operations that may be performed by the body unit of FIG. 1 to update inventory information;

FIG. 24 is a flowchart illustrating a sequence of operations that may be performed by the body unit of FIG. 1 to receive at least a portion of patient information and, in response, request additional patient information;

FIG. 25 is a flowchart illustrating a sequence of operations that may be performed by the body unit of FIG. 1 to communicate with an EMS agency, hospital and/or other entity.

It should be understood that the appended drawings are not necessarily to scale, presenting a somewhat simplified representation of various preferred features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments may have been enlarged, distorted or otherwise rendered differently relative to others to facilitate visualization and clear understanding.

### DETAILED DESCRIPTION

### Hardware and Software Environment

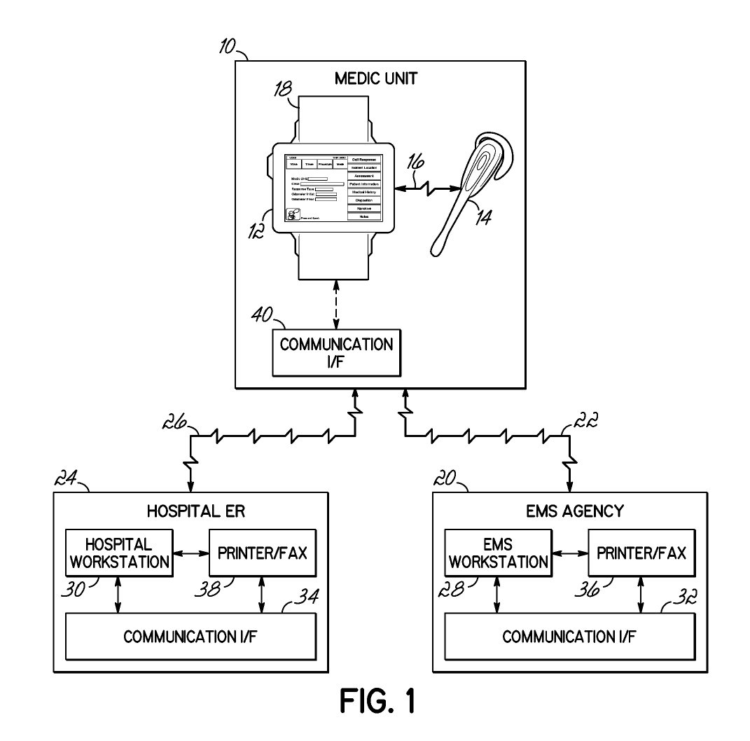

Turning to the drawings, wherein like numbers denote like parts throughout the several views, FIG. 1 is a diagrammatic illustration of an overview of a hardware environment for a documentation and communication system 10 consistent with embodiments of the invention. As illustrated in FIG. 1, the documentation and communication system 10 (hereinafter, “system” 10) includes a body unit 12 and a headset 14. The body unit 12, in some embodiments, is a body-worn touchscreen computing device that is configured to communicate with the headset as at 16 to convert speech input from a user (not shown) received by the headset 14 into machine readable input and to appropriately store, process, and/or perform an action in response to the speech input from the user. In specific embodiments, the body unit 12 is configured to store translated speech input, to communicate externally to retrieve data in response to translated speech input, to prompt a user to perform an action in response to speech input, to maintain an inventory of a medic unit and/or perform another action in response to speech input.

The headset 14 includes a microphone to receive the speech input and, in some embodiments, additionally includes a speaker. The headset 14 may be in communication with the body unit 12 through a wireless communication link 16 such as, for example, through a personal area network (e.g., Bluetooth). The body unit 12 may include a strap 18 such that the body unit 12 may be worn on a forearm of the user, while the headset 14 may be worn upon the ear of the user.

In some embodiments, the system 10 is in communication with an emergency medical services (“EMS”) agency 20 by way of a communications link 22. The system 10 may also be in communication with a destination, such as a hospital, or other care facility, 24, and in particular an emergency ward (e.g., more colloquially, an emergency “room”) by way of a communications link 26. It will be appreciated that communication links 22 and 26 may be wireless communications links, such as cellular network links, radio network links, or other wireless network links. The body unit 12 may also communicate with other entities, such as a police station, a dispatch station and/or a networked source of information. For example, the dispatch station may be a central station that provides dispatches to local medical units (e.g., an ambulance, a helicopter, a patient transport unit, and/or another medical services transportation unit). As such, the dispatch station may be a local 911-response center that sends out calls for emergencies to the EMS agency 20, the hospital 24 and/or other destinations.

The EMS agency 20 and the hospital 24 may be configured with at least one respective EMS workstation 28 and hospital workstation 30. The workstations 28, 30, in specific embodiments, are configured with, or otherwise in communication with, respective communication interfaces 32, 34 (illustrated as, and hereinafter, “communication I/Fs 32, 34”) as well as respective printers and/or fax machines 36, 38 (printers and/or fax machines illustrated as, and hereinafter, “printer/fax 36, 38”). The EMS workstation 28 may be configured to receive data from the body unit 12 in the form of reports. The EMS workstation 28 may be further configured to store that data and/or subsequently transmit that data to a regulatory agency. The EMS workstation 28 may also be configured to send patient, protocol, procedure, contraindications, and/or other information to the body unit 12, or to update tasks to be performed by the user of the body unit 12. Similarly, the hospital workstation 30 may be configured to receive data from the body unit 12. In specific embodiments, the hospital workstation 30 is configured to receive trip data from the body unit 12 as the user and patient are en route to that hospital. As such, the hospital workstation 30 may receive a portion (e.g., all or some) of the trip data for that trip. Additionally, the hospital workstation 30 may be configured to send patient, protocol and/or procedure information to the body unit 12, or to update tasks to be performed by the user of the body unit 12.

As illustrated, the system 10 may be in direct communication with the EMS agency 20 and the hospital 24 such that the body unit 12 communicates directly with the EMS agency 20, the hospital 24 and/or the respective workstations 28, 30 thereof. In alternative embodiments, the body unit 12 is in indirect communication with the EMS agency 20 and/or the hospital 24 through a separate communications interface 40. In specific embodiments, the body unit 12 and headset 14 may be worn by an EMT, a paramedic, or other emergency medical services technician while the communications I/F 40 may be disposed in a medical unit (not shown). Thus, data from the body unit 12 may be transmitted to the communication I/F 40, which may be in turn transmitted to the EMS agency 20 and/or hospital 24. In alternative embodiments, data from the body unit 12 is transferred directly to at least one of the workstations 28, 30 and/or printer/fax machines 36, 38 by physically connecting the body unit 12 to that workstation 28, 30 and/or printer/fax machine 36, 38. In specific alternative embodiments, data from the body unit 12 is transferred to or from at least one of the workstations 28, 30 and/or printer/fax machines 36, 38 through the universal serial bus standard.

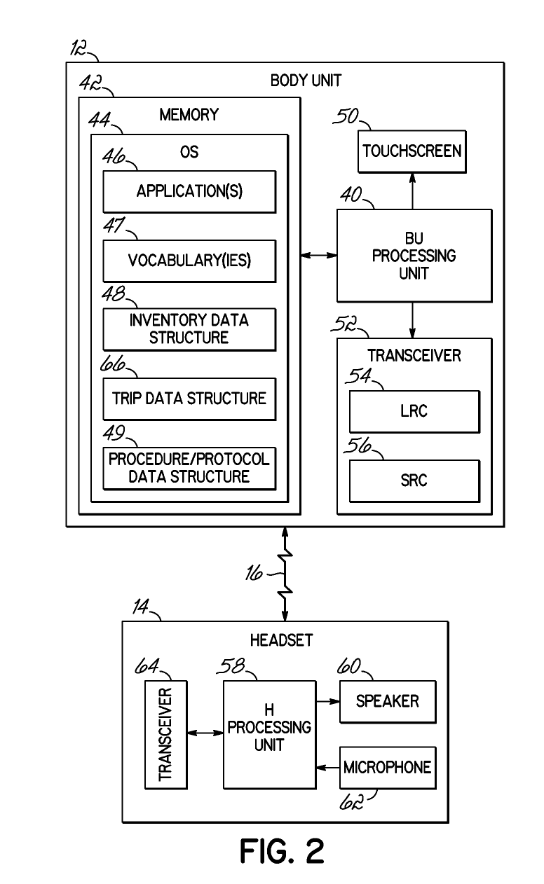

FIG. 2 is a diagrammatic illustration of the hardware environment of the body unit 12 and headset 14 of the system 10 of FIG. 1 consistent with embodiments of the invention. The body unit 12 includes at least one processing unit 40 (illustrated as, and hereinafter, “BU processing unit” 40) coupled to a memory 42. Each processing unit 40 may be one or more microprocessors, micro-controllers, field programmable gate arrays, or ASICs, while memory 42 may include random access memory (RAM), dynamic random access memory (DRAM), static random access memory (SRAM), flash memory, and/or another digital storage medium. The body unit 12 may be under the control of an operating system 44 and execute or otherwise relies upon various software applications, components, programs, files, objects, modules, etc. (illustrated as “application(s)” 46) consistent with embodiments of the invention. In specific embodiments, the operating system 44 is a Windows Embedded Compact operating system as distributed by Microsoft Corporation of Redmond, Wash. Alternatively, the operating system 44 may be a Linux based operating system. Also alternatively, the operating system 44 may be a Unix based operating system such as that distributed by Apple Inc. of Cupertino, Calif. The body unit 12 may be configured with at least one application 46 that, in turn, may rely on one or more vocabularies 47 to convert speech input of a user to machine readable input, generate a display representation on a touchscreen display 50, interface with the touchscreen 50 to determine user interaction, and/or communicate with the EMS agency 20 and/or hospital 24. Moreover, the body unit 12 may be configured with at least one application 46 that, in turn, may rely on one or more inventory data structures 48 to store data about inventory associated with the user, patient and/or medic unit. Additionally, the body unit 12 may be configured with at least one application 46 that, in turn, may rely on one or more procedure and/or protocol data structures 49 (illustrated as, and hereinafter, “procedure/protocol data structure” 49) to determine, display and/or walkthrough a procedure and/or protocol. In some embodiments, the procedure and/or protocol data structure 49 includes at least one guide to a protocol and/or a procedure, which in turn instructs a user how to perform a sequence of steps, operations and/or actions. The procedure and/or protocol may be a medical procedure, a medical protocol, an information gathering procedure, an inspection protocol and/or another procedure or protocol to perform a sequence of actions. In alternative embodiments, the body unit 12 does not include the touchscreen display 50 and instead includes a dedicated user input (e.g., such as an alphanumeric keypad) (not shown) and a non-touchscreen display (not shown).

The body unit 12 may include transceiver hardware 52 (e.g., in some embodiments, a transceiver), which in turn may include a long-range component 54 (illustrated as, and hereinafter, “LRC” 54) and/or a short-range component 56 (illustrated as, and hereinafter, “SRC” 56). In this manner, the body unit 12 may communicate with the EMS agency 20 and/or hospital 24 through the LRC 54 as well as communicate with the EMS agency 20, hospital 24 and/or headset 14 through the SRC 56.

In addition to illustrating one hardware environment of the body unit 12, FIG. 2 further illustrates a hardware environment of the headset 14 consistent with embodiments of the invention. In particular, the headset 14 may include at least one headset processing unit 58 (illustrated as, and hereinafter, “H processing unit” 58) in communication with a speaker 60 and microphone 62, and further coupled with a transceiver 64. The headset 14 may pick up speech input through the microphone 62, sample and/or otherwise digitize that speech input with the H processing unit 58, then send that sampled and/or digitized speech input to the body unit 12 through the transceiver 64. The body unit 12 may transmit at least one sound output to the headset 14 to play on the speaker 60 to interact with the user.

In some embodiments, the body unit 12 is configured to store data associated with at least one trip in a trip data structure 66. In some embodiments, the trip data structure 66 includes a database to organize data associated with a plurality of trips based upon a unique identification of the respective plurality of trips. In alternative embodiments, the trip data structure 66 includes a plurality of files, where each file is associated with a particular trip and includes information for that trip. Specifically, each file may be a word processing file as is well known in the art.

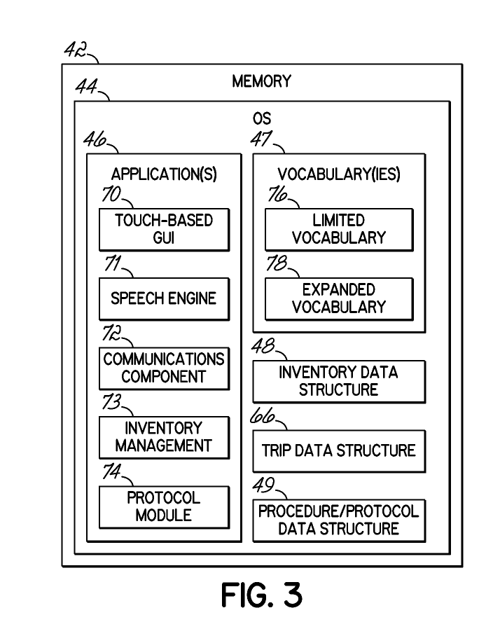

FIG. 3 is a diagrammatic illustration of the at least one application 46 and the at least one vocabulary 47 that may be disposed in the memory 42 of the body unit 12 consistent with embodiments of the invention. In particular, FIG. 3 illustrates that the at least one application 48 includes at least one touch-based graphic user interface 70 (illustrated as, and hereinafter, “touch-based GUI” 70), a speech engine 71, a communications component 72, an inventory management module 73 and/or a protocol module 74. In some embodiments, the touch-based GUI 70 is configured to interface with the touchscreen 50 and display images, screens, text and/or multimedia on the touchscreen 50. In particular, the touch-based GUI 70 is configured to provide a plurality of interactive screens to the user. The touch-based GUI 70 is configured to interface with the touchscreen 50 to determine interaction of the user with the touchscreen 50. For example, the touch-based GUI 70 may display a button on the touchscreen 50. In response to interaction with this button, the touch-based GUI 70 may pass that information for the body unit 12 to do something, such as display another screen.

The speech engine 71 may be a speech recognition engine configured to perform real-time conversion of speech input to machine readable input. The speech engine 71 may be configured to interface with the at least one vocabulary 47, which includes a limited vocabulary 76 and/or an expanded vocabulary 78. In some embodiments, the speech engine 71 interacts with the touch-based GUI 70 to determine which screen is being displayed. Depending upon the screen being displayed by the touch-based GUI 70 on the touchscreen 50, the speech engine 71 may convert speech input with the limited vocabulary 76 and/or the expanded vocabulary 78. For example, speech input regarding vital signs, times of events and medications may be converted with the limited vocabulary 76 while speech input regarding patient assessments, patient information and medical histories of a patient may be converted with the expanded vocabulary 78 depending on the possible responses or speech utterances that could be entered for the particular screen.

In alternative embodiments, the body unit 12 may capture data in another manner than speech input translation with the speech engine 71 without departing from the scope of the invention. In those embodiments, the body unit 12 may be configured to generate a display representation of a keyboard and detect interaction therewith. For example, and not intended to be limiting, the touch-based GUI 70 may be configured to display a representation of a keyboard on the touchscreen 50 and the body unit 12, in turn, may be configured to detect interaction with the keyboard on the touchscreen 50. In particular, the body unit 12 may be configured to detect interaction with the various keys of the keyboard display representation. Thus, a user may type in data to be entered and/or correct data that was entered. Similarly, the body unit 12 may be configured to capture handwriting. For example, and not intended to be limiting, the touch-based GUI 70 may be configured to display a representation of a handwriting capture area on the touchscreen 50 and the body unit 12, in turn, may be configured to detect interaction (e.g., by the user with a stylus, their finger and/or other implement) with the handwriting capture area on the touchscreen 50. In particular, the body unit 12 may be configured to detect interaction with the handwriting capture area and translate the interaction into data. Thus, a user may handwrite data to be entered and/or correct data that was entered. In specific embodiments, the keyboard and/or handwriting capture area may be controlled by software modules without departing from the scope of the invention. Furthermore, it will be appreciated that the handwriting capture area may be a display representation of a handwriting capture area, or the handwriting capture area may simply be a display representation of the current screen (e.g., the touchscreen 50 captures handwriting on the touchscreen 50 without the body unit 12 displaying a discrete handwriting capture area). In this manner, handwriting interaction with the touchscreen 50 may be automatically translated into data.

The communications component 72 may be configured to interface with the transceiver hardware 52 and/or communication interface 40 associated with the body unit to communicate with the EMS agency 20, the hospital 24 and/or another entity. Additionally, the communications component 72 may be configured to interface with the transceiver hardware 52 to communicate with headset 14.

The inventory management module 73 is configured to track inventory associated with the user, patient, and in particular the medic unit associated with the user. Advantageously, the body unit 12 may store a list of all inventory of the medic unit in the inventory data structure 48, which may be updated by the inventory management module 73 as that inventory is utilized, as that inventory is indicated to be unavailable (e.g., the user indicates that the inventory is broken, is used up or has been removed) and/or as inventory is added to the medic unit (e.g., as the user specifies that inventory has been added). Moreover, the inventory management module 73 may store the inventory used for a trip in the trip data structure 66. In this manner, a listing of inventory of the medic unit may be continually updated and later analyzed for billing purposes. For example, the inventory management module 73 may track the number of syringes, gauze and/or other medical instruments used during a trip and update the inventory data structure 48 and/or trip data structure 66 accordingly. Upon completion of the trip, the inventory data structure 48 and/or trip data in the trip data structure 66 may be transferred to the EMS agency 20 to determine the inventory used during that trip, and thus the amount to charge for the use of that inventory. In some embodiments, the inventory management module 73 is configured to alert the user when inventory is running low or otherwise unavailable. Additionally, the inventory management module 73 may be configured to induce the body unit 12 to communicate with the user and/or EMS agency 20 to re-order inventory that is running low or otherwise unavailable.

The protocol module 74 is configured to provide at least one image, audio prompt and/or multimedia presentation associated with a protocol and/or procedure to the user in response to speech input from the user. For example, and in specific embodiments, the speech engine 71 is configured to convert speech input into machine readable input. In response to the machine readable input, the protocol module 74 is configured to interface with the procedure/protocol data structure to display and/or guide the user through a protocol and/or procedure, such as a respective treatment protocol for a specific situation and/or a respective treatment procedure. The protocol module 74 may display and/or guide the user through a protocol and/or procedure through at least one image and/or multimedia presentation on the touchscreen 50 of the body unit, and/or through at least one audio prompt played through the speaker 60 of the headset 14.

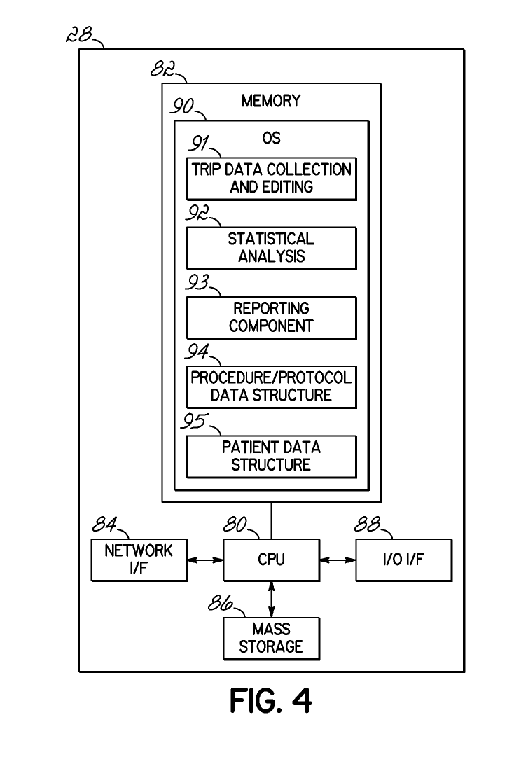

FIG. 4 is a diagrammatic illustration at least a portion of the hardware and software components of a workstation 28, 30 consistent with embodiments of the invention. In particular, FIG. 4 is a diagrammatic illustration of the hardware components of either the EMS workstation 28 or the hospital workstation 30. The EMS workstation 28 and/or hospital workstation 30, for purposes of this invention, may represent any type of computer, computing system, server, disk array, or programmable device such as a multi-user computer, single-user computer, handheld device, networked device, mobile phone, gaming system, etc. The EMS workstation 28 and/or hospital workstation 30 may be implemented using one or more networked computers, e.g., in a cluster or other distributed computing system.

The EMS workstation 28 and/or hospital workstation 30 typically includes at least one central processing unit (“CPU”) 80 coupled to a memory 82. Each CPU 80 may be one or more microprocessors, micro-controllers, field programmable gate arrays, or ASICs, while memory 82 may include random access memory (RAM), dynamic random access memory (DRAM), static random access memory (SRAM), flash memory, and/or another digital storage medium. As such, memory 82 may be considered to include memory storage physically located elsewhere in the EMS workstation 28 and/or hospital workstation 30, e.g., any cache memory in the at least one CPU 80, as well as any storage capacity used as a virtual memory, e.g., as stored on a mass storage device 86, a computer, or another controller coupled to computer through a network interface 84 (illustrated as, and hereinafter, “network I/F” 84) by way of a network.

The EMS workstation 28 and/or hospital workstation 30 may include the mass storage device 86, which may also be a digital storage medium, and in specific embodiments includes at least one hard disk drive. Additionally, mass storage device 86 may be located externally to the EMS workstation 28 and/or hospital workstation 30, such as in a separate enclosure or in one or more networked computers (not shown), one or more networked storage devices (including, for example, a tape drive) (not shown), and/or one or more other networked devices 26 (including, for example, a server) (not shown).

The EMS workstation 28 and/or hospital workstation 30 may also include peripheral devices connected to the computer through an input/output device interface 88 (illustrated as, and hereinafter, “I/O I/F” 88). In particular, the EMS workstation 28 and/or hospital workstation 30 may receive data from a user through at least one user interface (including, for example, a keyboard, mouse, and/or other user interface) (not shown) and/or output data to a user through at least one output device (including, for example, a display, speakers, and/or another output device) (not shown). Moreover, in some embodiments, the I/O I/F 88 communicates with a device that includes a user interface and at least one output device in combination, such as a touchscreen (not shown).

The EMS workstation 28 and/or hospital workstation 30 may be under the control of an operating system 90 and execute or otherwise relies upon various computer software applications, components, programs, files, objects, modules, etc., consistent with embodiments of the invention. In particular, the EMS workstation 28 may be configured with a trip data collection and editing software component 91, a statistical analysis software component 92, and a reporting software component 93. Moreover, the EMS workstation 28 and/or hospital workstation 30 may be configured with a protocol and/or procedure data structure 94 (illustrated as, and hereinafter, “protocol/procedure data structure” 94) and/or a patient data structure 95. The trip data collection and editing software component 91 may be used to gather documentation of a trip from the body unit 12 and edit that documentation. The statistical analysis software component 92 may be able to then perform statistical analysis of that documentation and the reporting software component 93 may be configured to report that edited documentation to a government agency.

In specific embodiments, the statistical analysis software component 92 is configured to mine the trip data to determine the response time of the user and/or medic unit to various locations, including from the dispatch call to the incident location and from the incident location to the destination. Moreover, the statistical analysis software component 92 may be configured to determine inventory used during the trip and the overall standard of care for the patient. In some embodiments, the statistical analysis software component 92 is configured to determine the average response times of a specific user and/or medic unit, as well as the average response times of all users and/or medic units of the entire EMS agency 20. Thus, the statistical analysis software component 92 may be configured to provide statistical data about users and/or medic units individually or as a whole.

The EMS workstation 28 and/or the hospital workstation 30 may include the protocol/procedure data structure 94 and/or patient data structure 95. In some embodiments, a user may request information about a protocol and/or procedure which is not present in the procedure/protocol data structure 49 of that body unit 12. As such, the body unit 12 may communicate with the EMS workstation 28 and/or the hospital workstation 30 to download that protocol and/or procedure information from the protocol/procedure data structure 94 of that respective workstation 28, 30. Similarly, the user may enter some information about the patient in the body unit 12 and request that the body unit query the patient data structure 95 for additional data of the patient from the patient data structure 95. In response to the query, additional data about the patient may be transmitted from the patient data structure 95 to the body unit 12, and the body unit 12 may use received patient data to fill in at least a portion of the trip data for the trip associated with that patient.

Those skilled in the art will recognize that the environments illustrated in FIGS. 1-4 are not intended to limit the present invention. In particular, while the body unit 12 includes a speech engine 71, in alternative embodiments the body unit 12 may include speech recognition hardware coupled to the BU processing unit 40 to translate speech input into machine readable input. Indeed, those having skill in the art will recognize that other alternative hardware and/or software environments may be used without departing from the scope of the invention. For example, the body unit 12 and headset 14 may include at least one power storage unit, such as a battery, capacitor and/or other power storage unit without departing from the scope of the invention.

Additionally, one having ordinary skill in the art will recognize that the environment for the body unit 12, headset 14, EMS workstation 28 and/or hospital workstation 30 is not intended to limit the scope of embodiments of the invention. For example, one having skill in the art will appreciate that the headset 14 may include memory and applications disposed therein to sample speech input picked up by the microphone 62 and/or communicate with the body unit 12. Similarly, one having skill in the art will appreciate that the EMS workstation 28 and/or hospital workstation 30 may include more or fewer applications than those illustrated, and that the hospital workstation 30 may include the same applications as those indicated are included in the EMS workstation 28. Similarly, one having skill in the art will appreciate that the software components of the EMS workstation 28 and/or hospital workstation 30 may be configured in alternate locations in communication with the body unit 12, such as across a network. As such, other alternative hardware environments may be used without departing from the scope of the invention.

The routines executed to implement the embodiments of the invention, whether implemented as part of an operating system or a specific application, component, program, object, module or sequence of instructions executed by the processing unit(s) or CPU(s) will be referred to herein as “computer program code,” or simply “program code.” The program code typically comprises one or more instructions that are resident at various times in various memory and storage devices in the body unit 12, EMS workstation 28 and/or hospital workstation 30, and that, when read and executed by one or more processing units or CPUs of the body unit 12, EMS workstation 28 and/or hospital workstation 30, cause that body unit 12, EMS workstation 28 and/or hospital workstation 30 to perform the steps necessary to execute steps, elements, and/or blocks embodying the various aspects of the invention.

While the invention has and hereinafter will be described in the context of fully functioning documentation and communication systems as well as computing systems, those skilled in the art will appreciate that the various embodiments of the invention are capable of being distributed as a program product in a variety of forms, and that the invention applies equally regardless of the particular type of computer readable signal bearing media used to actually carry out the distribution. Examples of computer readable signal bearing media include but are not limited to recordable type media such as volatile and nonvolatile memory devices, floppy and other removable disks, hard disk drives, optical disks (e.g., CD-ROM's, DVD's, etc.), among others, and transmission type media such as digital and analog communication links.

In addition, various program code described hereinafter may be identified based upon the application or software component within which it is implemented in a specific embodiment of the invention. However, it should be appreciated that any particular program nomenclature that follows is used merely for convenience, and thus the invention should not be limited to use solely in any specific application identified and/or implied by such nomenclature. Furthermore, given the typically endless number of manners in which computer programs may be organized into routines, procedures, methods, modules, objects, and the like, as well as the various manners in which program functionality may be allocated among various software layers that are resident within a typical computer (e.g., operating systems, libraries, APIs, applications, applets, etc.), it should be appreciated that the invention is not limited to the specific organization and allocation of program functionality described herein.

### Software Description and Flows

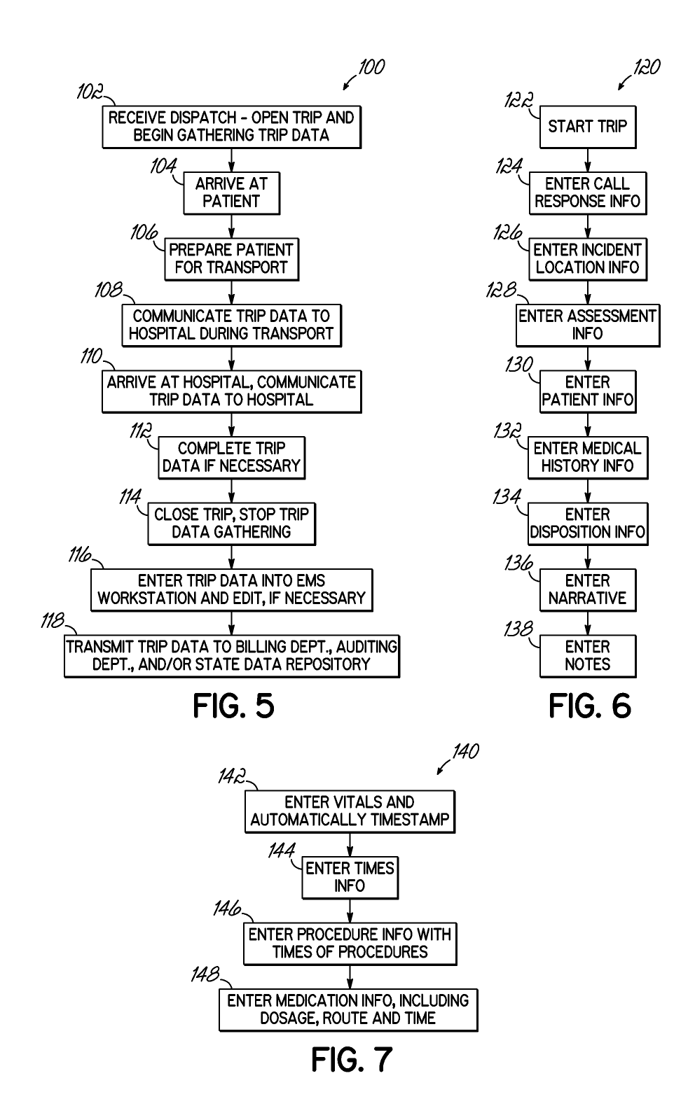

FIG. 5 is a flowchart 100 illustrating a sequence of steps during which a user may be dispatched to a patient to render transport for and/or emergency medical services to that patient. FIG. 5 also illustrates gathering trip data consistent with embodiments of the invention. In particular, a user may receive a dispatch to a patient and, in response to receiving the dispatch, open the trip and begin gathering trip data (block 102). The user may then arrive at the location of the patient (block 104) and prepare the patient for transport to a hospital (block 106). During transport of the patient to the hospital, the user may gather additional trip data and communicate that trip data to a hospital (block 108).

Upon arrival at the hospital, trip data that has not already been communicated to the hospital may be communicated to the hospital (block 110), trip data may be completed, if necessary (block 112), and the trip may be closed (thus halting trip data gathering) (block 114). In particular, the user may, in blocks 102 through 112, gather or enter some or all of the following trip data: information about the dispatch call, a location of the patient, an assessment of the patient, patient information (including medical history information and disposition information regarding the patient), a narrative of treatment of the patient and/or trip, notes about the patient and/or trip, vital signs of the patient, procedures performed on the patient, times associated with the patient and/or trip as well as medications administered to the patient. Upon return to an EMS agency, the user may enter the trip data into an EMS workstation and edit that trip data, if necessary (block 116). The user may then transmit that edited trip data to a billing department, an auditing department and/or a state data repository that may receive that trip data (block 118).

FIG. 6 is flowchart 120 illustrating a sequence of steps to enter trip data with a body unit and headset consistent with embodiments of the invention. In specific embodiments, the trip data illustrated in flowchart 120 is entered through the headset as speech input then translated by the body unit using an expanded vocabulary consistent with embodiments of the invention. In particular, the user may interact with the body unit (e.g., through a touchscreen of the body unit and/or through speech input translated by the body unit to machine readable input) to start a trip in response to a dispatch call (block 122) and enter call response information (block 124). The user may also enter incident location information based upon the information in the dispatch call and/or based on the scene at the incident location (block 126). In response to an initial consultation and/or examination of the patient, an assessment of the patient may be entered (block 128) along with patient information (block 130). If known, medical history information of the patient may also be entered (block 132). Disposition information associated with the patient may also be entered (block 134). In addition to specified information, the user may enter a narrative about the trip and/or the treatment of the patient (block 136). The user may also enter notes that are related to the trip and/or patient (block 138).

FIG. 7 is flowchart 140 illustrating a sequence of steps to enter trip data with the body unit and headset consistent with embodiments of the invention. In specific embodiments, the trip data illustrated in flowchart 140 is entered through the headset as speech input then translated by the body unit using a limited vocabulary consistent with embodiments of the invention. In particular, the user may enter vital signs of the patient, and, in response to converting the speech input of the vital signs to machine readable input, the body unit may automatically timestamp the vital signs (block 142). In some embodiments, the blood pressure, pulse, temperature, and/or respiration rate of the patient may be taken at multiple times, each instance of which may be timestamped. The user may then enter times associated with the trip data, such as the time of the dispatch call, the time the user and/or medic unit was notified of the call, the time the user and/or medic unit started en route to the scene of the incident location, time the user and/or medic unit arrived at the scene, the time the user arrived at the patient, the time the patient left the scene, the time the patient arrived at the destination, and/or the time the medic unit and/or user was placed back in service to receive another dispatch call (block 144). In addition to vital signs and times, the user may enter information associated with procedures performed on the patient (block 146). In particular, the procedure information is associated with procedures performed on the patient at the scene, procedures performed on the patient en route to the destination and/or procedures performed on the patient before the unit and/or patient leaves the destination after having transferred to the patient to the destination. When the user enters procedure information, the user may indicate a time that procedure was performed. In some embodiments, the user also enters medication information, including an identification of the medication administered to the patient, the dosage of the medication, the route of the medication and/or the time the medication was administered (block 148).

Thus, and with reference to FIGS. 5-6, the user and/or medic unit associated therewith may receive a dispatch call for emergency medical services. The user may enter information about the call and scene of the incident location en route and, upon arrive, enter additional information about the incident location. The user may arrive at the patient and conduct a preliminary assessment, then prepare the patient for transport. Assessment information and/or patient information may be entered, along with medical history information of the patient and the disposition of the patient, if known. At the incident location or en route to the destination (e.g., a hospital), vital signs of the patient, time information associated with the trip, procedure information, or medical information may also be entered. Trip information may be transmitted, in advance, to the destination as well as communicated to a workstation of the destination. The user may make notes or otherwise enter a narrative about the trip to complete trip data, then close the trip to stop trip data gathering. The trip data may be entered into an EMS workstation and edited, if necessary, then sent to a billing department, auditing department and/or state data repository.

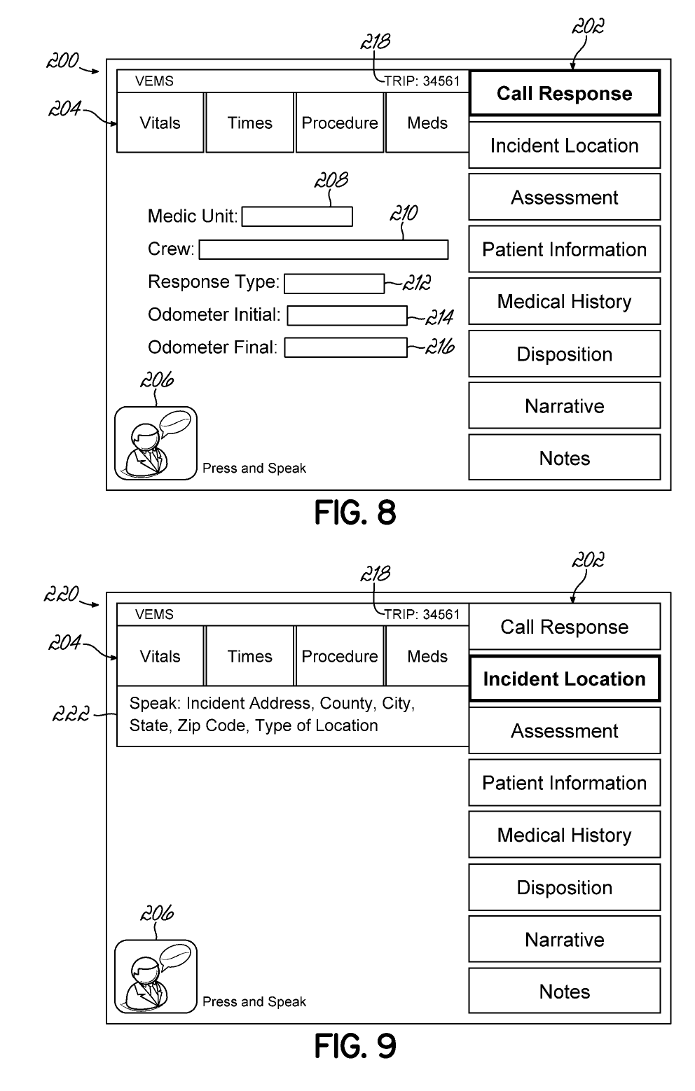

Consistent with embodiments of the invention, FIGS. 8-19 illustrate a plurality of screens that may be generated by a touch-based GUI associated of the body unit to interact with a user to gather trip data. In some embodiments, FIGS. 8-19 illustrate a plurality of screens that suit the workflow of a user in which to enter dispatch call information, incident location information, assessment information, patient information, medical history information, patient disposition information, narrative information, notes, patient vital signs, trip time information, procedure information, and/or medication information. It will be appreciated that each of the screens may be selected by interfacing with the touchscreen to touch a corresponding screen name associated with a screen and/or through translated speech input that specifies that screen.

FIG. 8 is an illustration of a call response screen 200 in which the user may enter dispatch call information. Moreover, FIG. 8 illustrates a trip screen selection menu 202, a treatment screen selection menu 204, and a speech conversion button 206. In particular, the user may select a trip screen to view by interacting with the trip screen selection menu 202. In specific embodiments, the body unit includes a touchscreen and the user may select a trip screen to view by interacting with (e.g., touching) a corresponding screen name in the trip screen selection menu 202. In specific embodiments, the body unit may translate speech input specifying the trip screen to select into machine readable input and, in response to that machine readable input, select a trip screen corresponding to that machine readable input. For example, the user may say “call response” and the body unit may display the call response screen 200. Similarly, the user may select a treatment screen to view by interacting with a corresponding screen name in the treatment screen selection menu 204 and/or through speech input.

In some embodiments, the user enters trip information through speech input picked up by the headset and translated by the headset or body unit, or a combination of the headset and body unit, into machine readable input. The user enables the conversion of speech input associated with trip data to machine readable input associated with trip data by interacting with the speech conversion button 206. In some embodiments, the user enables the conversion of speech input to machine readable input during the time that the speech conversion button 206 is held. In alternative embodiments, the user enables the conversion of speech input to machine readable input for a specified period of time after the speech conversion button 206 is interacted with and/or until the speech input from the user is to “stop.” In some embodiments, information for each of the trip screens may be translated by a speech engine with an expanded library, while information for each of the treatment screens may be translated by the speech engine with a limited vocabulary as discussed herein.

In some embodiments, each screen is associated with at least one field. Information for these fields may be input through speech input. The body unit is configured to convert at least a portion of the speech input or utterances into machine readable input (e.g., text) and operably input that machine readable input into the selected field. More specifically, and with reference to the call response screen 200 of FIG. 8, information for the medic unit field 208 may be input by the user selecting the medic unit field 208 through touch (e.g., touching the medic unit field 208) or speaking “medic unit” to select that field to select that field when the speech conversion button 206 has been interacted with. In that later case, the body unit may translate at least a portion of the speech input following “medic unit” into information about the medic unit associated with that user. In a similar manner, the user may enter information associated with the crew, type of response, initial odometer reading and/or final odometer reading in the respective crew field 210, response type field 212, initial odometer field 214 and/or final odometer field 216. One having skill in the art will appreciate that additional information may be entered in the call response screen 200, and thus the invention should not be limited to the input of the call response information disclosed in the illustrated embodiments.

As illustrated in FIG. 8, each screen may include a trip counter 218 that indicates the specific trip for which trip information is being entered. In this manner, information for a plurality of trips may be stored in the body unit, and information for each of the plurality of trips may be associated with a respective number indicated by the trip counter 218. Upon the end of data collection for a trip, the trip counter 218 may be incremented.

FIG. 9 is an illustration of an incident location screen 220 in which the user may enter information about an incident location in an incident location field 222. In particular, the user may select the incident location field 222 and enter information about the scene of the incident, including the address, county, city, state, zip code, and/or type of location associated with that incident location. In some embodiments, the incident location field 222 is automatically selected in response to interacting with the speech conversion button 206 on the incident location screen 220. Thus, and with reference to the incident location screen 220, the user may interact with the speech conversion button 206 and automatically select the incident location field 222 to enter incident location information. One having skill in the art will appreciate that additional information may be entered in the incident location field 242, and thus the invention should not be limited to the input of the incident location information disclosed in the illustrated embodiments.

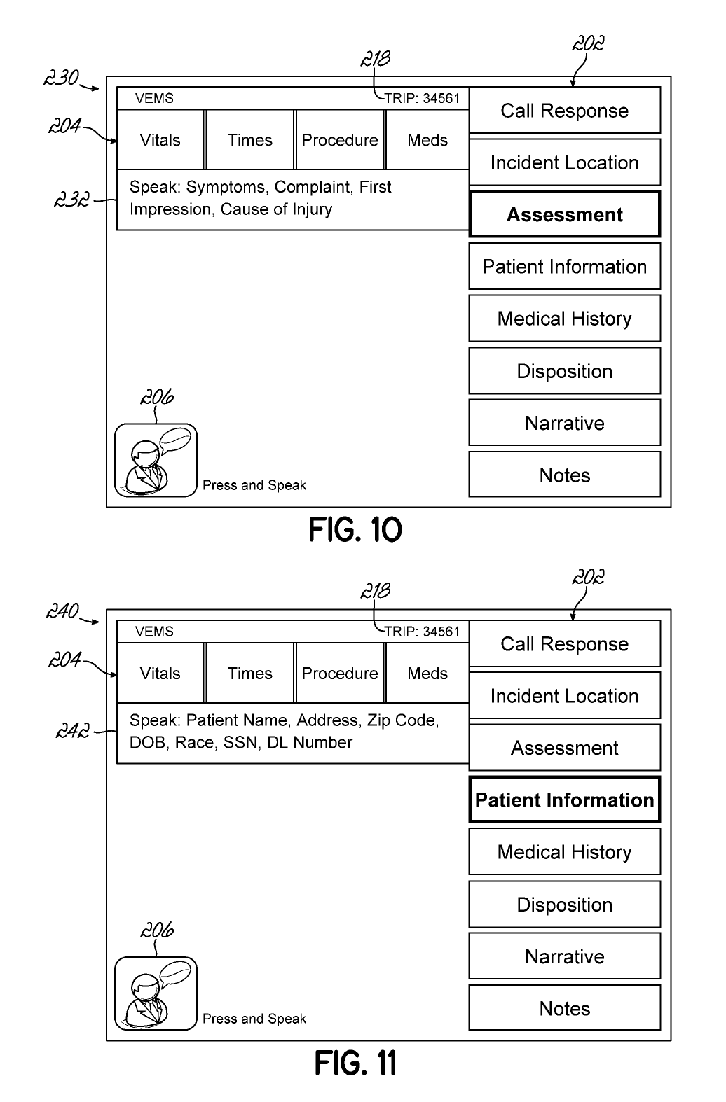

FIG. 10 is an illustration of an assessment screen 230 in which the user may enter information about an assessment of a patient in an assessment field 232. In particular, the user may select the assessment field 232 and enter information about a symptom of the patient, a complaint of the patient, a first impression of the patient and/or the cause of injury to the patient. In some embodiments, the assessment field 232 is automatically selected in response to interacting with the speech conversion button 206 on the assessment screen 230. One having skill in the art will appreciate that additional information may be entered in the assessment field 242, and thus the invention should not be limited to the input of the assessment information disclosed in the illustrated embodiments.

FIG. 11 is an illustration of a patient information screen 240 in which the user may enter information about an assessment of a patient in a patient information field 242. In particular, the user may select the patient information field 242 and enter information about the patient, including their name, address, city, state zip code, date of birth, race, social security number and/or a driver's license number associated with that patient. In some embodiments, the patient information field 242 is automatically selected in response to interacting with the speech conversion button 206 on the patient information screen 240. One having skill in the art will appreciate that additional information may be entered in the patient information field 242, and thus the invention should not be limited to the input of the patient information disclosed in the illustrated embodiments.

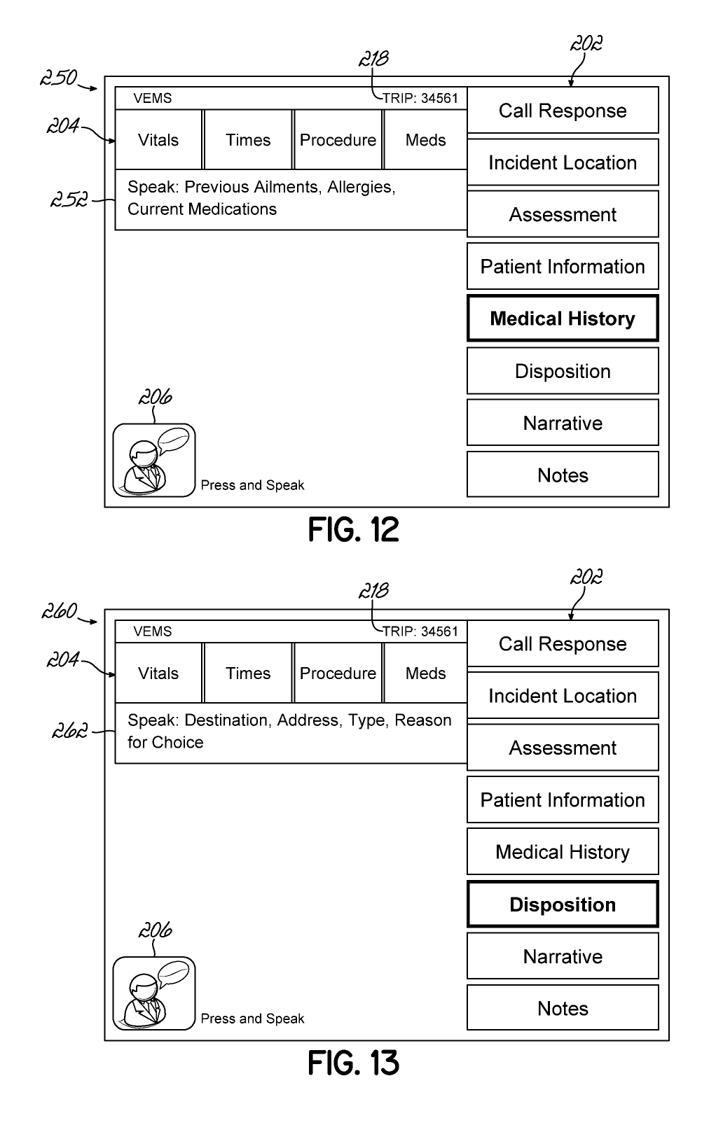

FIG. 12 is an illustration of a medical history screen 250 in which the user may enter information about a medical history of the patient in a medical history field 252. In particular, the user may select the medical history field 252 and enter information about the medical history of the patient, including previous ailments, allergies and/or current medications of the patient. In some embodiments, the medical history field 252 is automatically selected in response to interacting with the speech conversion button 206 on the medical history screen 250. One having skill in the art will appreciate that additional information may be entered in the medical history field 252, and thus the invention should not be limited to the input of the medical history information disclosed in the illustrated embodiments.

FIG. 13 is an illustration of a patient disposition screen 260 in which the user may enter information about a disposition of the patient in a patient disposition field 262. In particular, the user may select the patient disposition field 262 and enter information about the disposition of the patient, including the destination of the patient, the address for the destination (e.g., including the county, city, state and/or zip code of the destination address) and/or the reason for the choice of the destination (e.g., destination is closest, destination specializes in this particular type of injury, etc.). In some embodiments, the patient disposition field 262 is automatically selected in response to interacting with the speech conversion button 206 on the patient disposition screen 260. One having skill in the art will appreciate that additional information may be entered in the patient disposition field 262, and thus the invention should not be limited to the input of the patient disposition information disclosed in the illustrated embodiments.

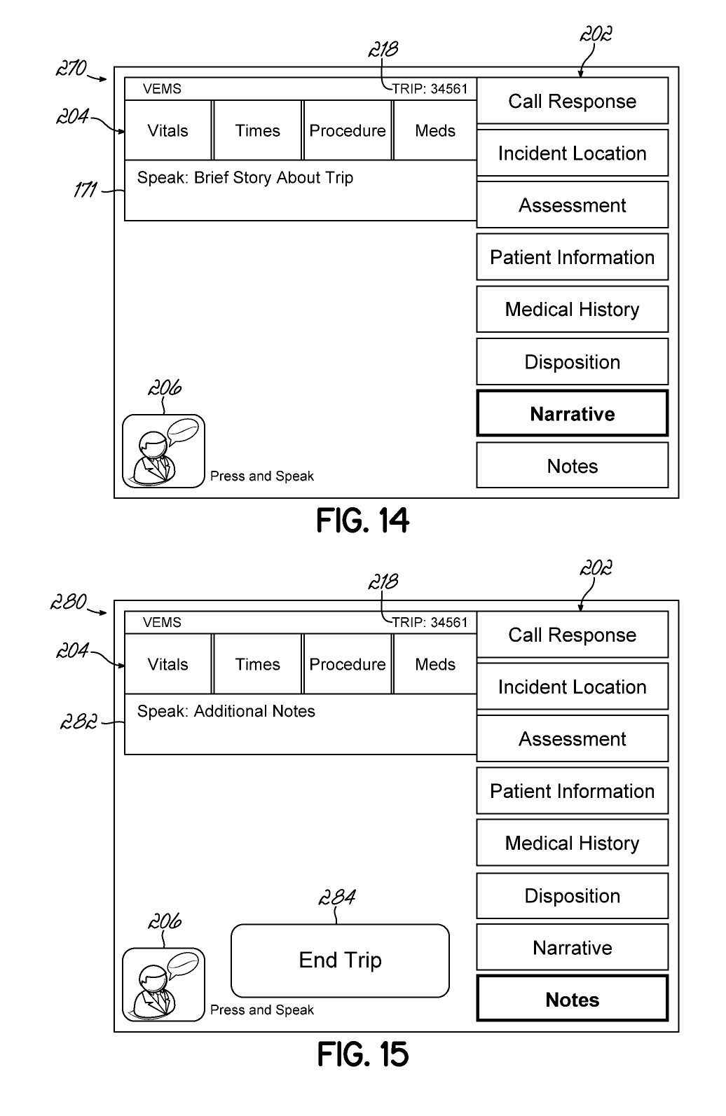

FIG. 14 is an illustration of a narrative screen 270 in which the user may enter a narrative of the trip in a narrative field 272. In particular, the user may select the narrative field 272 and enter a narrative of the trip, including a brief story of the trip. In some embodiments, the narrative field 272 is automatically selected in response to interacting with the speech conversion button 206 on the narrative screen 270. One having skill in the art will appreciate that additional information may be entered in the narrative field 272, and thus the invention should not be limited to the input of the narrative information disclosed in the illustrated embodiments.

FIG. 15 is an illustration of a notes screen 280 in which the user may enter notes in a notes field 282. In particular, the user may select the notes field 282 and enter notes, including notes about the trip, notes about the patient, notes about the medic unit, notes about supplies and/or any other notes the user feels are appropriate to include. In some embodiments, the notes field 282 is automatically selected in response to interacting with the speech conversion button 206 on the notes screen 280.

In addition to the speech conversion button 206, the notes screen 280 includes the end trip button 284. In response to interacting with the end trip button 284, data collection for the trip is completed and the information associated with that trip is stored in a trip data structure. In some embodiments, in response to interacting with the end trip button 284, the user is unable to enter information for a trip through the body unit, as that trip is considered “closed.” As such, subsequent information is associated with a new number indicated on the trip counter 218, and thus a new trip. One having skill in the art will appreciate that additional information may be entered in the notes field 282, and thus the invention should not be limited to the input of the notes information disclosed in the illustrated embodiments.

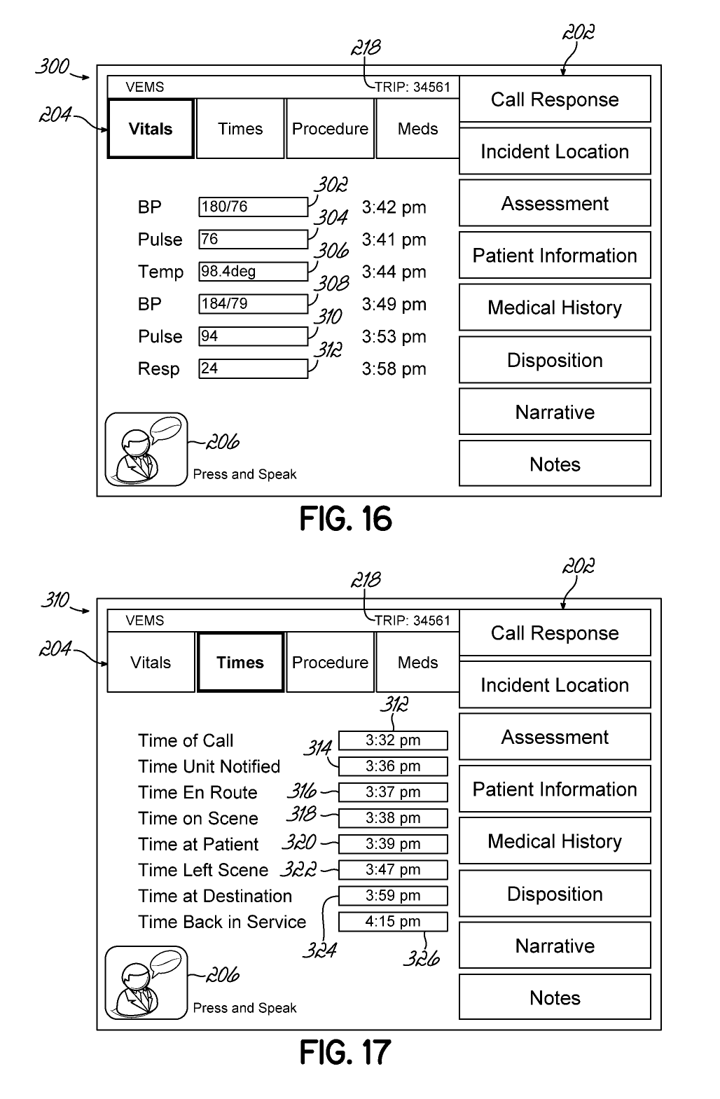

FIG. 16 is an illustration of a vitals screen 300 in which the user may enter vital signs of the patient. In particular, the user may enter the patient's blood pressure, pulse, temperature and/or respiration rate on the vitals screen 300. More specifically, and with reference to the vitals screen 300 of FIG. 16, information for a blood pressure field 302 may be input by the user selecting the blood pressure field 302 through touch (e.g., touching the blood pressure field 302) or speaking “blood pressure” to select that field when the speech conversion button 206 has been interacted with. In the later case, the body unit may translate at least a portion of the speech input following “blood pressure” into information about the blood pressure of a patient. In a similar manner the user may enter information associated with the pulse, temperature and/or respiration rate of the patient in the respective at least one pulse field 304, temperature field 306 and/or respiration rate field 308. As vital signs are entered in each field 302-308, the information may be timestamped. In some embodiments, each of the fields 302-308 may be selected multiple times and vital signs entered. Thus, only the most recent vital signs are illustrated, while previous vital signs may be stored in the trip data structure. One having skill in the art will appreciate that additional information may be entered in the vitals screen 300, and thus the invention should not be limited to the input of the vital signs information disclosed in the illustrated embodiments.

FIG. 17 is an illustration of a times screen 310 in which the user may enter times associated with the trip. More specifically, and with reference to the times screen 310 of FIG. 17, information associated with a time of the dispatch call may be input by the user selecting the time of call field 312 through touch (e.g., touching the time of call field 312) or speaking “time of call” to select that field when the speech conversion button 206 has been interacted with. In the later case, the body unit may translate at least a portion of the speech input following “time of call” into information about the time of the dispatch call. In a similar manner, the user may enter times associated with the time the user and/or medic unit was notified of the call, the time the user and/or medic unit started en route to the scene of the incident location, time the user and/or medic unit arrived at the scene, the time the user arrived at the patient, the time the patient left the scene, the time the patient arrived at the destination, and/or the time the medic unit and/or user was placed back in service to receive another dispatch call on the times screen 310. More specifically, the user may enter information associated with the time of the dispatch call, the time the user and/or medic unit was notified of the call, the time the user and/or medic unit started en route to the scene of the incident location, time the user and/or medic unit arrived at the scene, the time the user arrived at the patient, the time the patient left the scene, the time the patient arrived at the destination, and/or the time the medic unit and/or user was placed back in service to receive another dispatch call in the respective time unit notified field 314, time en route field 316, time on scene field 318, time at patient field 320, time left scene field 322, time at destination field 324 and/or time back in service field 326. One having skill in the art will appreciate that additional information may be entered in the times screen 310, and thus the invention should not be limited to the input of the time information disclosed in the illustrated embodiments.

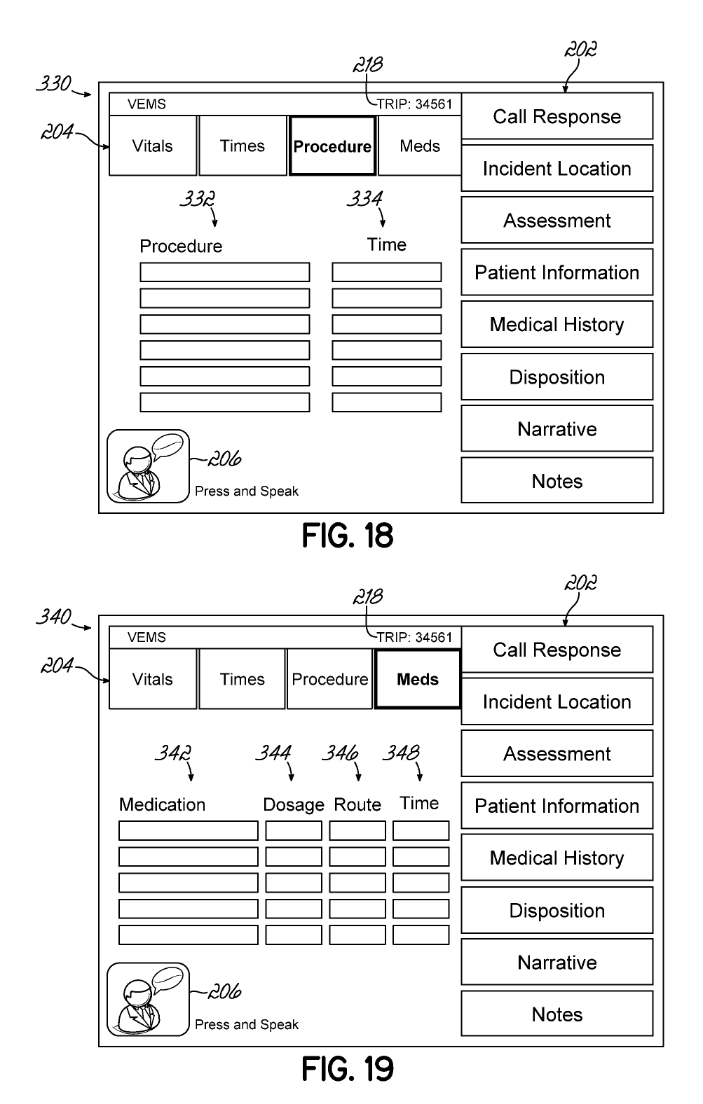

FIG. 18 is an illustration of a procedures screen 330 in which the user may enter information about a plurality of procedures, and time associated therewith, in the respective procedure fields 332 and procedure time fields 334. More specifically, and with reference to the procedures screen 330 of FIG. 18, information associated with a procedure may be input by the user selecting one of the procedure fields 332 through touch (e.g., touching a procedures field 332) or speaking “procedure” to select the first open procedure field 332 when the speech conversion button 206 has been interacted with. In the later case, the body unit may translate at least a portion of the speech input following “procedure” into information about the procedure. In a similar manner, the user may enter a time associated with the procedure (e.g., a time at which the procedure was performed) by either selecting a corresponding time field 334 for that procedure field 332 or simply speaking the time. In some embodiments, the procedure fields 332 and time fields 334 display only the six most recent procedures and respective times. Thus, only the most recent procedures and respective times are illustrated, while previous procedures and respective times may be stored in the trip data structure. One having ordinary skill in the art will appreciate that more or fewer procedures and respective times may be displayed without departing from the scope of the invention.

FIG. 19 is an illustration of a medications screen 340 in which the user may enter information about a plurality of medications, as well as dosages, routes and/or times associated therewith, in the respective medication fields 342, dosage fields 344, route fields 346 and/or medication time fields 348. More specifically, and with reference to the medications screen 340 of FIG. 19, information associated with a medication may be input by the user selecting one of the medication fields 342 through touch (e.g., touching a medication field 342) or speaking “medication” to select the first open medication field 342 when the speech conversion button 206 has been interacted with. In the later case, the body unit may translate at least a portion of the speech input following “medication” into information about the medication. In a similar manner, the user may enter a dosage, route and/or time associated with the procedure (e.g., a time at which the procedure was performed) by either selecting a corresponding dosage field 344, route field 346 and/or time field 348 for that medication field 342, or simply speaking the respective dosage, route and/or time. In some embodiments, the medication fields 342, dosage fields 344, route fields 346 and time fields 348 display only the five most recent medications and respective dosages, routes and/or times. Thus, only the most recent medications and respective dosages, routes and/or times are illustrated, while previous medications and respective dosages, routes and/or times may be stored in the trip data structure. One having ordinary skill in the art will appreciate that more or fewer medications and respective dosages, routes and/or times are illustrated may be displayed without departing from the scope of the invention.

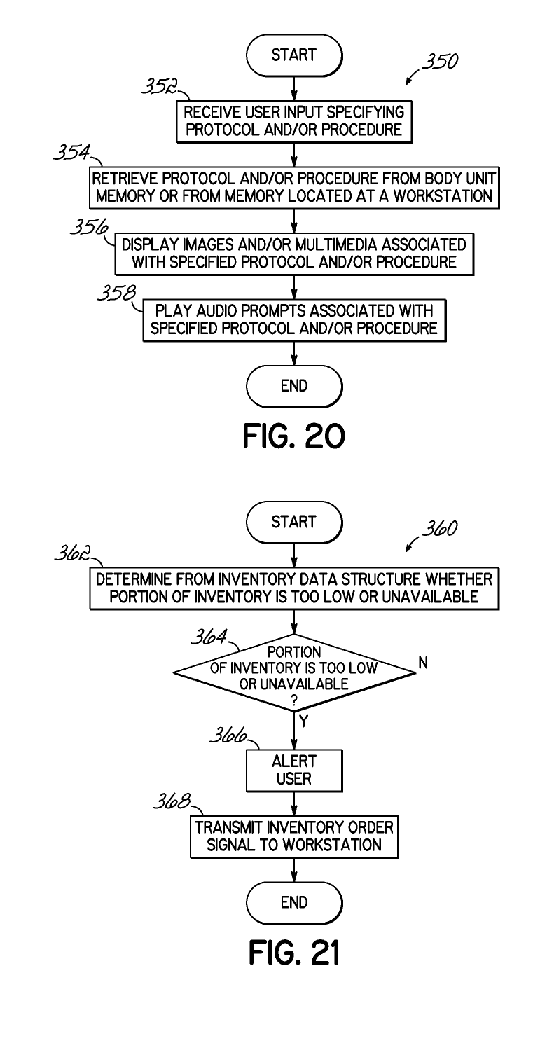

FIG. 20 is a flowchart 350 illustrating a sequence of operations that may be performed by the body unit to display images and/or a multimedia presentation, and/or play audio prompts, of a protocol and/or procedure consistent with embodiments of the invention. In particular, the body unit may receive user input specifying a protocol and/or procedure to display (block 352). In specific embodiments, the user specifies a protocol and/or procedure to display through speech input, which is converted into machine readable input to cause the body unit to display that protocol and/or procedure. Thus, the body unit may attempt to retrieve the protocol and/or procedure from the memory of the body unit (e.g., a protocol/procedure data structure resident on the memory of the body unit) and/or from memory located at a workstation in communication with the body unit (e.g., a protocol/procedure data structure resident on an EMS workstation, a hospital workstation and/or another memory in communication with the body unit) (block 354). In response to retrieving the protocol and/or procedure, the body unit may display images and/or multimedia presentations associated with the specified protocol and/or procedure (block 356). In specific embodiments, the body unit guides the user through the protocol and/or procedure by displaying the images and/or multimedia presentation in a particular sequence. In those embodiments, the user may advance to relevant portions of the images and/or multimedia presentation through speech input and/or by interfacing with the touchscreen of the body unit (e.g., initial steps of the procedure may have already been performed, and the user may wish to advance to portions of the protocol and/or procedure that they require more information about). In some embodiments, audio prompts associated with the specified protocol and/or procedure are also played on the speaker of the headset of the user (block 368). As such, the user may not have to refer to the body unit and may be guided through the protocol and/or procedure through the audio prompts.

FIG. 21 is a flowchart 360 illustrating a sequence of operations that may be performed by the body unit to determine whether, upon start-up or upon a request from a user, there is a portion of the inventory that is too low or unavailable. In some embodiments, in response to start-up of the body unit and/or a request from the user associated with that body unit, the body unit queries an inventory data structure to determine if at least a portion of inventory (e.g., tools, needles, medication, etc.) is too low or otherwise unavailable (e.g., the portion of inventory is broken, sent off for repair, etc.) (block 362). When a portion of the inventory is too low or otherwise unavailable (“Yes” branch of decision block 364) the body unit may alert the user (block 366) and transmit a signal to order that portion of inventory (e.g., an “inventory order signal”) to an EMS agency, and in particular to an EMS workstation of the EMS agency (block 368). When a portion of the inventory is not too low or otherwise unavailable (“No” branch of decision block 366) the sequence of operations may end.

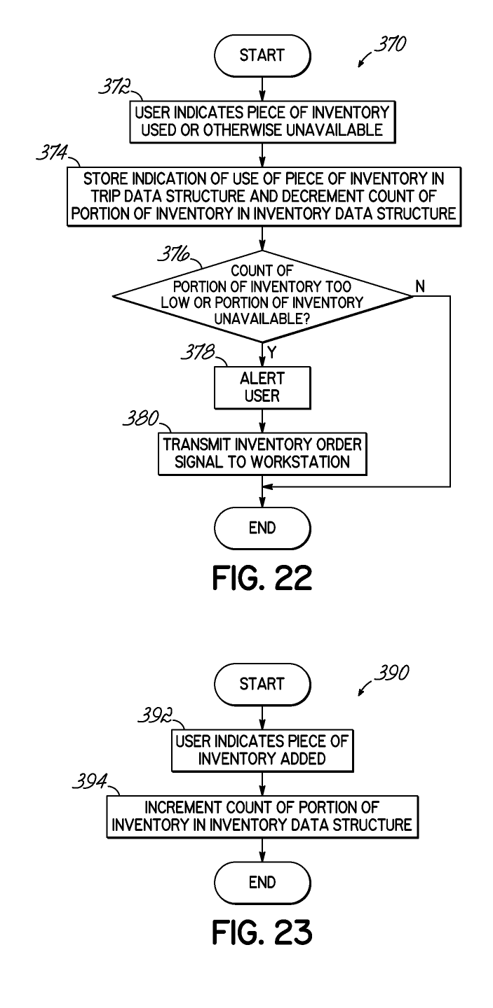

FIG. 22 is a flowchart 370 illustrating a sequence of operations that may be performed by the body unit to determine whether, upon use of a piece of the inventory or and indication that a piece of inventory is unavailable, that portion of the inventory is too low or unavailable. Upon use of a piece of inventory (e.g., use of a tool, a needle, a medication, etc.), the user may indicate that the piece of inventory was used (block 372). Alternately, upon inspection of an inventory (e.g., such as an inspection device, a defibrillator), the user may indicate that a piece of the inventory is unavailable (block 372). As such, the indication associated with that piece of inventory may be stored in a trip data structure and a count of a portion of inventory associated with that piece of inventory (e.g., for example, the inventory may indicate that a portion of the inventory includes one type of tools, and a count associated with that portion of the inventory may indicate that there are four tools, or four pieces, in the portion of the inventory) may be decremented (block 374). The body unit may then determine whether the count of the portion of the inventory is too low or whether the portion of the inventory is otherwise unavailable (block 376). When the count of the portion of the inventory is too low or when the portion of the inventory is otherwise unavailable (“Yes” branch of decision block 376) the body unit may alert the user (block 378) and transmit a signal to order that portion of inventory (e.g., an “inventory order signal”) to an EMS agency, and in particular to an EMS workstation of the EMS agency (block 380). When the count of the portion of the inventory is not too low and when the portion of the inventory is otherwise available (“No” branch of decision block 376) the sequence of operations may end.

FIG. 23 is flowchart 390 illustrating a sequence of operations that may be performed by the body unit to update inventory information consistent with embodiments of the invention. The user may interface with the body unit indicate that a piece of inventory has been added (block 392) and, in response to this indication, a count of a portion of the inventory associated with that piece of inventory may be incremented (block 394).

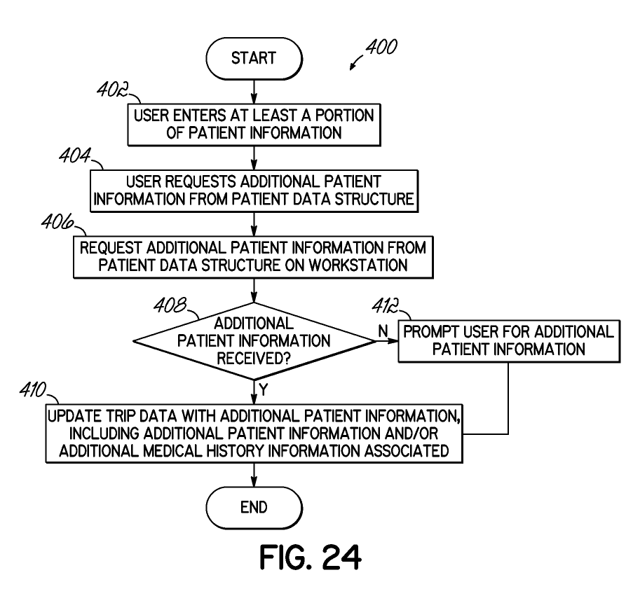

FIG. 24 is a flowchart 400 illustrating a sequence of operations that may be performed by the body unit to receive at least a portion of patient information and, in response, retrieve additional patient information. The user may enter at least a portion of patient information (block 402) and also request additional patient information from a patient data structure (block 404). As such, the body unit may issue a request for additional patient information from the patient data structure, such as a patient data structure in the memory of a workstation, and more particularly an EMS workstation or hospital workstation (block 406). In some embodiments, the request for the additional patient information includes some of the portion of patient information previously entered by the user such that the workstation can utilize that portion of patient information to retrieve additional patient information. When the body unit receives additional patient information (“Yes” branch of decision block 408) the body unit may update the trip data with the additional patient information (block 410). In some embodiments, this additional patient information includes patient information that is entered in the patient information screen 240 or the medical history screen 250. Returning to block 408, when the additional patient information is not received (“No” branch of decision block 408) the body unit may prompt the user for the additional patient information (block 412) or otherwise indicate that the additional patient information has not been received.

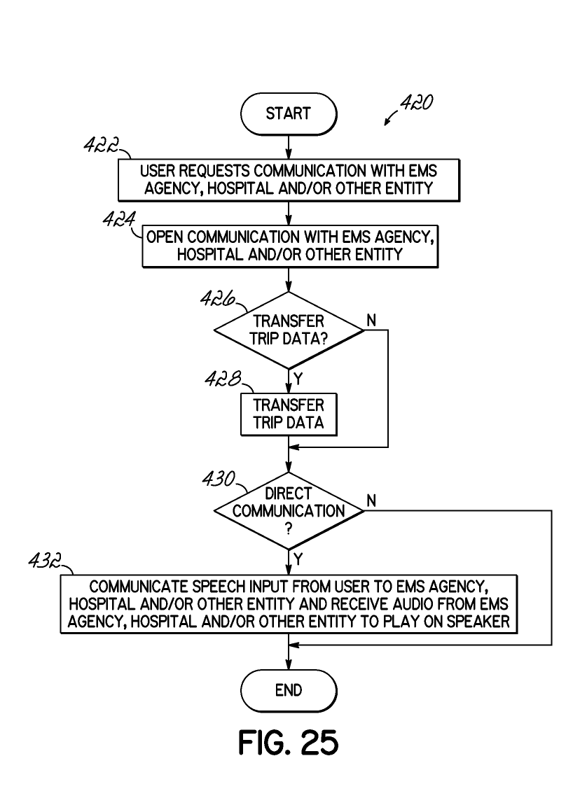

FIG. 25 is a flowchart 420 illustrating a sequence of operations that may be performed by the body unit to communicate with an EMS agency, hospital and/or other entity consistent with embodiments of the invention. In some embodiments, the user requests to communicate with an EMS agency, hospital and/or other entity (block 422). In specific embodiments, the user requests to communicate with the EMS agency, hospital and/or other entity by interfacing with the body unit through speech input to transfer trip data and/or open direct communication between the user and that entity. As such, the body unit may open communications with the EMS agency, hospital and/or other entity through a transceiver and/or communication I/F (block 424). When the body unit determines that the user has requested the transfer of trip data (“Yes” branch of decision block 426) the body unit transfers the trip data to the EMS agency, hospital and/or other entity (block 428). When the body unit determines that the user has not requested the transfer of trip data (“No” branch of decision block 426) or after transferring trip data (block 428), the body unit may determine whether the user requested to open a direct line of communication with the EMS agency, hospital and/or other entity (block 430). When the body unit determines that the user requested to open a direct line of communication with the EMS agency, hospital and/or other entity (“Yes” branch of decision block 430) the body unit may communicate speech input from the user to that EMS agency, hospital and/or other entity and receive audio from the EMS agency, hospital and/or other entity to play on the speaker of the headset (block 432). When the body unit determines that the user has not requested to open a direct line of communication with the EMS agency, hospital and/or other entity (“No” branch of decision block 430) the sequence of operations may end.

Thus, throughout the embodiments, a system consistent with embodiments of the invention provides for a body unit in communication with a headset, the body unit configured to translate speech input from the user into machine readable input. The body unit is configured to store that machine readable input and/or perform some operation in response to that machine readable input. The body unit may be provided with a touchscreen to display a plurality of screens to capture trip data for emergency medical services. The trip data may be stored or sent to an entity in communication with that body unit. Moreover, patient information may be retrieved from that entity. The body unit is further configured to display a guide to a protocol and/or procedure for the user, monitor inventory for the user, and help the user communicate with the entity. In particular, the body unit is configured to communicate trip data and/or provide audio between the user and the entity. Thus, in specific embodiments, the system, which may include the body unit and headset, provides a hands-free ability to perform EMS trip sheet documentation, to address checklist procedures, or to make queries of certain protocols or procedures using voice, all while tending to a patient. The system may provide a unique multi-modal (e.g., touchscreen and speech input) interaction directed to the emergency process that emergency service technicians work through during a dispatch call in order to provide them the ability to document and communicate in a hands-free manner. Advantageously, it is believed that embodiments of the invention provide documentation and communication in a fraction of the current time that is required, and further does not significantly interfere with patient care while also providing increased documentation accuracy.

In some embodiments, and in a similar manner as requesting protocols and/or procedures, the system provides a user with a contraindication list through voice queries. Advantageously, this may eliminates the need for various protocol texts, references, and pocket guides. For example, the user may speak into the headset and ask for a list of contraindications to a specific drug. The body unit may translate the speech input into a query for a list of contraindications to that drug. If the body unit does not have that list in its memory, the body unit may transmit that query to the EMS workstation, hospital workstation and/or other data structure. The EMS workstation, hospital workstation and/or other data structure may process the query and transmit this list of contraindications to the body unit. When the body unit has the list of contraindications, the body unit may display that list on the display and/or translate the list into an audio list and play that list on the speaker of the headset. Advantageously, this may result in the user not having to reference paper documents while treating the patient.

In some embodiments, the system may be used to perform an inventory and/or inspection of equipment. For example, the body unit may be configured to illustrate checklists for inventory and/or inspection. The user may then interact with the checklists through speech input or the touchscreen display. For example, the body unit may inquire as to whether a user has specific inventory, or an acceptable inventory, by questioning the user about the inventory through the speaker on the headset. The user may respond “Yes,” instructing the body unit to store an affirmative response that there is specific and/or acceptable inventory.

While embodiments of the invention have been illustrated by a description of the various embodiments and the examples, and while these embodiments have been described in considerable detail, it is not the intention of the applicants to restrict or in any way limit the scope of the appended claims to such detail. Additional advantages and modifications will readily appear to those skilled in the art. Thus, embodiments of the invention in broader aspects are therefore not limited to the specific details, representative apparatus and method, and illustrative example shown and described. For example, embodiments of the invention, in broader aspects, are not limited to field documentation and care of patients by emergency medical personnel. Embodiments of the invention may additionally be used by physicians, nurses, hospital staff, hospital volunteers and/or other medical caregivers. It will further be appreciated by one having skill in the art that embodiments of the invention may be used in separate fields that require documentation and thus, for example, be extended to field service of systems, inspection documentation, maintenance documentation and/or plant operations. Additionally, any of the blocks of the above flowcharts may be deleted, augmented, made to be simultaneous with another, combined, or be otherwise altered in accordance with the principles of the present invention. Accordingly, departures may be made from such details without departing from the spirit or scope of applicants' general inventive concept.

Other modifications will be apparent to one of ordinary skill in the art. Therefore, the invention lies in the claims hereinafter appended.

What is claimed is:

1. A method of documenting information with a wearable computing device of the type that includes a processing unit and a touchscreen display, the method comprising:
displaying at least one screen on the touchscreen display;
selecting a field on the at least one screen in which to enter data;
receiving speech input from a user;
converting the speech input into machine readable input; and
displaying the machine readable input in the field on the at least one screen.

2. The method of claim 1, wherein converting the speech input to machine readable input further comprises:
converting the speech input to machine readable input with a speech recognition engine.

3. The method of claim 1, wherein converting the speech input to machine readable input further comprises:
converting the speech input to machine readable input with at least one of a limited library and an expanded library.

4. The method of claim 3, wherein the speech input associated with at least one of vital signs of a patient, times associated with a trip, procedures performed on the patient or medications administered to the patient is converted into machine readable documentation with the limited library.

5. The method of claim 1, wherein converting the speech input into machine readable input includes:
detecting user interaction with a speech conversion button displayed on the touchscreen display; and
in response to the user interaction, converting the speech input into machine readable input.

6. The method of claim 5, wherein converting the speech input into machine readable input occurs during the detected user interaction with the speech conversion button displayed on the touchscreen display.

7. The method of claim 1, wherein selecting the field on the at least one screen in which to enter data includes:
detecting user interaction with the touchscreen display to select the field; and
in response to the user interaction, selecting the field.

8. The method of claim 1, wherein selecting a field on the at least one screen in which to enter data includes:
receiving speech input;
converting the speech input into machine readable input; and
from the machine readable input, determining the field to select to enter data and selecting the field.

9. The method of claim 1, further comprising:
communicating the machine readable input to at least one computing device.

10. The method of claim 1, wherein the speech input includes speech input selected from the group consisting of information about a dispatch to a patient, information about a location of the patient, information about an assessment of the patient, personal information about the patient, information about a medical history of the patient, information about a disposition of the patient, a narrative of treatment of a trip, notes about the trip, vital signs of the patient, procedures performed on the patient, times associated with the trip, medications administered to the patient, and combinations thereof.

11. The method of claim 1, further comprising:
storing the machine readable input with a unique identifier of a trip associated with that machine readable input.

12. The method of claim 1, further comprising;
communicating the machine readable input to a state data repository configured to receive the machine readable input.

13. The method of claim 1, further comprising:
displaying a protocol to the user.

14. The method of claim 13, wherein displaying a protocol to the user includes:
in response to user interaction with the wearable computing device to view the protocol, requesting the protocol from a computing device in communication with the wearable computing device, wherein displaying the protocol to the user is performed in response to receiving the protocol.

15. The method of claim 1, further comprising:
monitoring an inventory associated with the wearable computing device.

16. The method of claim 15, further comprising:
in response to an indication that a piece of inventory has been used, updating the inventory associated with the wearable computing device.

17. The method of claim 1, wherein the machine readable input is at least a portion of patient information, the method further comprising:
requesting additional patient information based on the at least a portion of patient information.

18. The method of claim 17, further comprising:
receiving the additional patient information; and
automatically displaying the additional patient information in a second field on the at least one screen.

19. A documentation and communication system, comprising:
a headset for capturing speech input from a user; and
a wearable computing device in communication with the headset and configured to convert the speech input into machine readable input, the wearable computing device including:
at least one processing unit;
a memory including at least one library, wherein the wearable computing device converts the speech input into machine readable input with the at least one library;
a display configured to display the machine readable input as it is converted from the speech input; and a wireless transceiver to transmit the machine readable input to at least one computing device;

20. The documentation and communication system of claim 19, wherein the at least one library further comprises:
a limited library; and
an expanded library,
wherein the wearable computing device converts the speech input associated with at least one of vital signs, times, procedures, and medications into machine readable documentation with the limited library.

21. The documentation and communication system of claim 19, wherein the wearable computing device and headset are configured to be worn by an emergency medical technician to electronically document care of a patient in real-time.

22. The documentation and communication system of claim 19, wherein the display is a touchscreen display.

23. The documentation and communication system of claim 19, wherein the system provides multimodal data entry through the touchscreen and the conversion of speech input to machine readable input.

24. A documentation and communication system, comprising:
a headset for capturing speech input from a user; and
a wearable computing device in communication with the headset, the wearable computing device including:
at least one processing unit;
a touchscreen display; and
memory including program code, the program code configured to be executed by the at least one processing unit to document information by displaying at least one screen on the touchscreen display, selecting a field on the at least one screen in which to enter data, receiving the speech input from a user, converting the speech input into machine readable input, and displaying the machine readable input in the field on the at least one screen.

25. The documentation and communication system of claim 24, wherein the program code is further configured to convert the speech input to machine readable input with a speech recognition engine.

26. The documentation and communication system of claim 24, wherein the program code is further configured to convert the speech input to machine readable input with at least one of a limited library and an expanded library.

27. The documentation and communication system of claim 24, wherein the speech input associated with at least one of vital signs of a patient, times associated with a trip, procedures performed on the patient or medications administered to the patient is converted into machine readable documentation with the limited library.

28. The documentation and communication system of claim 24, wherein the program code is further configured to detect user interaction with a speech conversion button displayed on the touchscreen display and, in response to the user interaction, convert the speech input into machine readable input.

29. The documentation and communication system of claim 28, wherein the program code is further configured to convert the speech input into machine readable input during the detected user interaction with the speech conversion button displayed on the touchscreen display

30. The documentation and communication system of claim 24, wherein the program code is further configured to detect user interaction with the touchscreen display to select the field and, in response to the user interaction, select the field.

31. The documentation and communication system of claim 24, wherein the speech input is first speech input, wherein the machine readable input is first machine readable input, and wherein the program code is further configured to receive second speech input, convert the second speech input into second machine readable input, and, from the second machine readable input, determine the field to select to enter data and selecting the field

32. The documentation and communication system of claim 24, wherein the program code is further configured to communicate the machine readable input to at least one computing device.

33. The documentation and communication system of claim 24, wherein the speech input includes speech input selected from the group consisting of information about a dispatch to a patient, information about a location of the patient, information about an assessment of the patient, personal information about the patient, information about a medical history of the patient, information about a disposition of the patient, a narrative of treatment of a trip, notes about the trip, vital signs of the patient, procedures performed on the patient, times associated with the trip, medications administered to the patient, and combinations thereof.

34. The documentation and communication system of claim 24, wherein the program code is further configured to store the machine readable input with a unique identifier of a trip associated with that machine readable input.

35. The documentation and communication system of claim 24, wherein the program code is further configured to communicate the machine readable input to a state data repository configured to receive the machine readable input.

36. The documentation and communication system of claim 24, wherein the program code is further configured to display a protocol to the user.

37. The documentation and communication system of claim 36, wherein the program code is further configured to request the protocol from a computing device in communication with the wearable computing device in response to user interaction with the wearable computing device to view the protocol and display the protocol to the user in response to receiving the protocol.

38. The documentation and communication system of claim 24, wherein the program code is further configured to monitor an inventory associated with the wearable computing device.

39. The documentation and communication system of claim 38, wherein the program code is further configured to update the inventory associated with the wearable computing device in response to an indication that a piece of inventory has been used.

40. The documentation and communication system of claim 24, wherein the machine readable input is at least a portion of patient information, and wherein the program code is further configured to request additional patient information based on the at least a portion of patient information.

41. The documentation and communication system of claim 40, wherein the program code is further configured to receive the additional patient information and automatically display the additional patient information in a second field on the at least one screen.
